(19) United States
(12) Patent Application Publication
(10) Pub. No.: US 2018/0103974 A1
(43) Pub. Date: Apr. 19, 2018

(54) THROMBECTOMY CATHETER

(71) Applicant: Cook Medical Technologies LLC, Bloomington, IN (US)

(72) Inventors: Thomas A. Osborne, Bloomington, IN (US); Charles W. Agnew, West Lafayette, IN (US)

(21) Appl. No.: 15/729,879

(22) Filed: Oct. 11, 2017

Related U.S. Application Data
(60) Provisional application No. 62/408,289, filed on Oct. 14, 2016.

(51) Int. Cl. A61B 17/3203; A61M 25/00; A61B 17/22

(52) U.S. Cl. CPC A61B 17/32037; A61M 2025/109; A61B 17/22032; A61M 25/0071

(57) ABSTRACT

A medical device for use in extraction of emboli from a body vessel is described. The medical device includes a tubular member comprising a proximal end and extending to a distal end, and a collection member having a collapsed configuration and an expanded configuration and being movable therebetween, the collection member having an apical end disposed to the distal end of the tubular member and extending to a basal end defining a body portion therebetween, the apical and basal ends having a second lumen formed therethrough and in fluid communication with the tubular member, the collection member comprising at least one port formed through the body portion.

### BACKGROUND

The present disclosure relates generally to a medical device for using in removing thromboemboli from the vasculature of a patient in need of treatment. More particularly, the present disclosure relates to a catheter having a generally cone-shaped collecting member to gather emboli from a body vessel.

The presence of blood clots or thrombus in the vascular system, if left untreated, can cause serious and potentially life-threatening disease. Thrombi within the vasculature can form as a result from a variety of causes, such as trauma, disease, surgery, stagnant flow of blood, among other reasons.

Typically, a thrombus present in an arterial blood vessel tends to migrate, whereupon it becomes known as an embolus or a thromboembolus. Embolic migration continues in the direction of flow from a large diameter artery to smaller diameter arteries. The thrombus continues to flow with the blood until it becomes lodged against the vessel wall and is unable to advance. In some instances, the thrombus partially or completely blocks blood flow through the artery thereby preventing blood from reaching the tissue disposed downstream of the thrombus. Denying blood flow for an extended period of time can result in damage or death of the tissue beyond this point. Additionally, thrombi in the venous system can migrate to the lungs and become a pulmonary embolus, which can be fatal. In other instances, thrombi can migrate into the cerebral circulation and cause stroke and death.

Thrombus removal, or thrombectomy, may be performed in a variety of ways. For example, the clot may be dissolved through chemical lysis using drugs. Used alone, though, lysis by drugs can be a relatively slow process, taking hours or even days to complete. Additionally, the drugs utilized in clot lysis cause the blood to thin.

Thrombectomy may also be performed using mechanical devices. Typically, these devices are inserted into a patient's vasculature and delivered to a treatment site over a guide wire using the Seldinger technique. In some cases, mechanical devices may not be strong enough to adequately capture and remove a thrombus because these devices must be small and flexible in order to negotiate the tortuous anatomy where thrombi are likely to be found.

It has been a challenge to develop a flexible device for capture and removal of thromboemboli from a body vessel of a patient.

### SUMMARY

According to a first aspect of the present invention, a medical device for use in extraction of emboli from a body vessel is provided. The medical device includes a tubular member having a proximal end and extending to a distal end, the tubular member having a first lumen formed therethrough. The medical device also includes a collection member having a collapsed configuration and an expanded configuration, the collection member being movable therebetween, the collection member having an apical end disposed at the distal end of the tubular member and extending to a basal end defining a body portion therebetween. The basal end is located distal the apical end. the apical and basal ends having a second lumen formed therethrough and in fluid communication with the tubular member, the collection member comprising at least one port formed through the body portion

In another embodiment, a medical device assembly for use in extraction of emboli from a body vessel is provided. The medical device assembly includes an outer tubular member having a first proximal end and extending to a first distal end, the outer tubular member having a passageway extending therethrough and an opening at the first distal end. The assembly also includes an inner tubular member having a second proximal end and extending to a second distal end, the inner tubular member having a second lumen formed therethrough, and a collection member having a collapsed configuration and an expanded configuration, the collection member being movable therebetween, the collection member having an apical end disposed to the second distal end of the inner tubular member and extending to a basal end defining a body portion therebetween. The basal end is located distal the apical end. The apical and basal ends have a third lumen formed therethrough and in fluid communication with the inner tubular member. The collection member includes at least one port formed through the body portion, wherein the collection member is axially movable between a first position and a second position, the collection member being in the collapsed configuration and disposed within the outer tubular member in the first position, the basal end of the collection member being axially extended from the outer tubular member in the second position.

Further aspects, features, and advantages of the invention will become apparent from consideration of the following description and the appended claims when taken in connection with the accompanying drawings.

### BRIEF DESCRIPTION OF THE DRAWINGS

Preferred embodiments of the present invention are described below, by way of example only with reference to the accompanying drawings in which:

FIG. 1 is a side view of a catheter and a substantially conical collection member in an expanded state, in accordance with one embodiment of the present invention;

FIG. 2 is a side view of the catheter of FIG. 1 in a second, collapsed configuration;

FIG. 3 is a side view of a catheter in accordance with another embodiment of the present invention;

FIG. 4 is a side view of a collection member of a catheter in accordance with another embodiment of the present invention;

FIG. 5 is a view of catheter assembly in accordance with another embodiment of the present invention;

FIG. 6 is a view of catheter assembly including an infusion catheter in accordance with another embodiment of the present invention;

FIGS. 7A, 7B, 7C, and 7D are cross sectional views of catheter assemblies in accordance with embodiments of the present invention;

FIG. 8 is a side cross sectional view of a catheter assembly with the inner catheter in an expanded state, in accordance with another embodiment of the present invention;

FIG. 9 is a side cross sectional view of a catheter assembly with the inner catheter in a collapsed state, in accordance with another embodiment of the present invention;

FIG. 10 is an end view of a collection member in a collapsed state in accordance with an embodiment of the present invention;

FIG. 11 is a perspective view of a catheter assembly including a collection member having multiple infusion ports in accordance with the principles of the present invention;

FIG. 12 is another perspective view of the catheter assembly of the embodiment of FIG. 11;

FIG. 13 is a side view of a device according to another embodiment of the present disclosure being used in a procedure for collecting emboli;

FIG. 14 is a schematic view of a method of disrupting a thrombus and capturing emboli in accordance with the principles of the present invention;

FIG. 15 is a perspective view of a catheter assembly including a wire guide constructed in accordance with the principles of the present disclosure;

FIG. 16A is a partial cutaway side view of a device in accordance with another embodiment of the present disclosure;

FIG. 16B is a cross-sectional view of the device of FIG. 16A; and

FIG. 16C is another side view of the device of FIG. 16A.

### DETAILED DESCRIPTION

It is to be understood that the figures are schematic and do not show the various components to their actual scale. In many instances, the figures show scaled up components to assist the reader. The following detailed description of the preferred embodiments will make clear the preferred arrangement, size relationships and manner of using the components shown herein.

In this description, when referring to a deployment assembly or a medical device, the term distal is used to refer to an end of a component which in use is furthest from the surgeon during the medical procedure, including within a patient. The term proximal is used to refer to an end of a component closest to the surgeon and in practice in or adjacent an external manipulation part of the deployment or treatment apparatus.

“Substantially” or derivatives thereof as used herein will be understood to mean significantly or in large part. The terms “substantially” or “about” used herein with reference to a quantity includes variations in the recited quantity that are equivalent to the quantity recited, such as an amount that is equivalent to the quantity recited for an intended purpose or function.

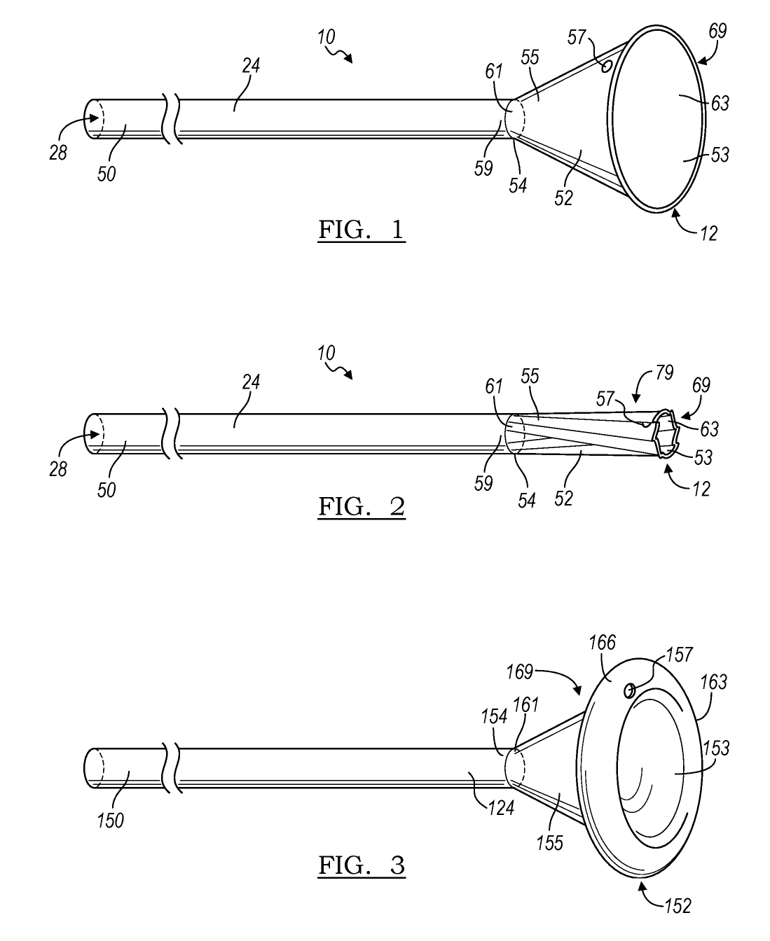

FIG. 1 depicts a catheter 10 having a proximal portion 50 and a catheter body or tubular member 24 that extends distally from proximal portion 50 to distal portion 59, which terminates at distal end 54. The catheter can generally be made of flexible polymers commonly used for such purposes in the medical device art, such as polyurethane, polyvinylchloride (PVC), and silicone. Catheter 10 can include a lumen 28 extending therethrough. Lumen 28 may be a first lumen of the device.

The catheter 10 further includes a collection member 52 which is located distal to distal end 54 of the catheter body 24. The collection member 52 is generally conical in shape and includes a body portion 55 which extends from apical end 61 to basal end 63. As shown in FIG. 1, the apical end 61 is located proximally relative to the basal end 63, and the basal end 63 is distal the apical end 61. The collection member 52 may have a lumen 53, which may be the second lumen of the device, running through the body portion from the apical end 61 to basal end 63. The collection member 52 also includes an outer surface 51 and an inner surface 63. The inner surface 63 surrounds lumen 53 and is opposite the outer surface 51.

The collection member 52 is so shaped such that it can act as a funnel for trapping embolic particles in a body vessel. The collection member 52 may have a largest diameter at its basal end 63 which is substantially equal to the diameter of the body vessel to which it is intended to be deployed, such that it forms a seal with the vessel wall in the expanded state, or may be designed with a diameter slightly larger than that of the vessel so as to ensure more complete sealing. In another embodiment, the largest diameter of the collection member may be somewhat smaller than the diameter of the vessel to which it is to be deployed in the instance that a seal is not necessary for proper operation of the device.

The collection member 52 may also comprise at least one port 57 formed through the body portion 55 at a point between the apical end 61 and the basal end 63. The port 57 is not coincident with the apical end 61, but is in fluid communication with it via the lumen 53. The collection member 52 may be formed with the port 57 through it, or the collection member 52 may be formed with no port and instead the port 57 may be introduced by a process such as punching. In some embodiments, the collection member 52 may have a plurality of ports 57 formed therethrough. The ports 57 are shown as being generally circular in shape, but other shapes, such as elliptical, rectangular, or other polygonal ports are possible. In some embodiments, the ports 57 may simply be linear slits or curved slits.

The collection member 52 may be in fluid communication with the tubular member 24 via the apical end 61. Therefore, in such an embodiment, the first lumen 28 is in fluid communication with the second lumen 53. The port 57, therefore, is in fluid communication with the first lumen 28 via the second lumen 53.

As shown in FIG. 1, the collection member 52 is in a first configuration which is expanded configuration 69. In the expanded configuration 69, the collection member 52 takes on a truncated conical shape and widens from the apical end 61 to basal end 63. The collection member 52 may be constructed of a pliable or flexible material such that it is biased into its expanded configuration 69 absent an external constraining force.

FIG. 2 illustrates the device 10 of FIG. 1 in a collapsed configuration. To facilitate radial collapse and expansion, the collection member 52 can be made of a flexible biocompatible material. In some embodiments, the collection member 52 can be made of a low durometer material such as but not limited to elastomers, rubbers, polyurethanes, and silicones.

FIG. 3 illustrates another embodiment of a device 110 having tubular member 124 and collection member 152. The collection member 152 in this embodiment includes an outer lip 166 at basal end 163, which may be a thickened ridge that runs circumferentially around the basal end 163 of collection member 152. In the illustrated embodiment, the port 157 is formed through the lip 166, but in other embodiments, a port may be formed through another part of the body member 155 (not shown.)

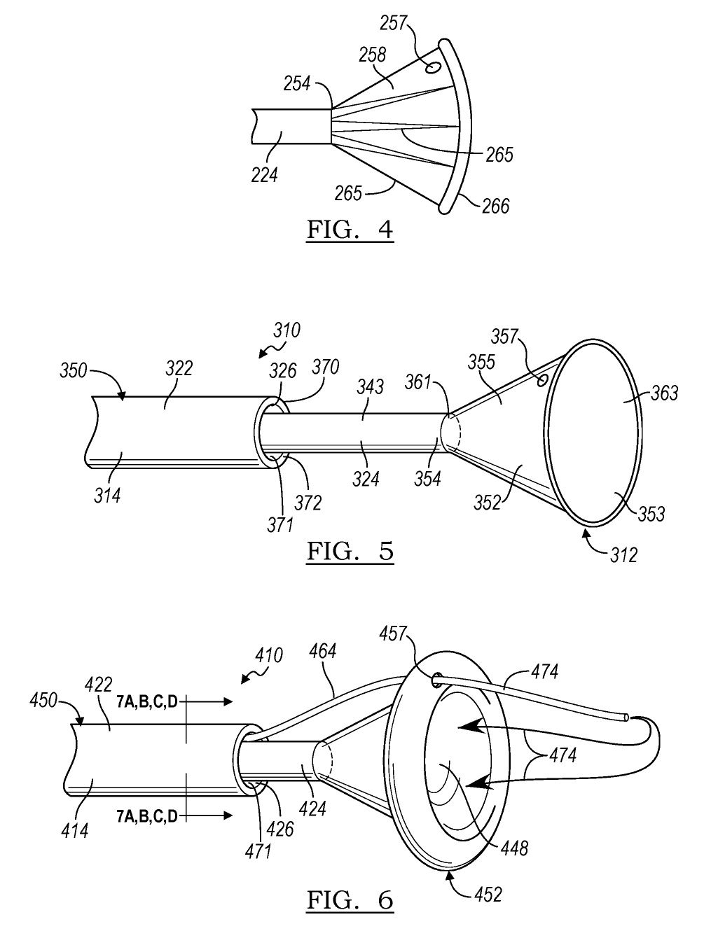

FIG. 4 shows an alternative design for a collection member 252 for use in a device 210. In this embodiment, the collection member 252 is a self-expanding element, which includes a covering 258 supported by a frame 265. The covering 258 may be made of a resilient material which readily expands and folds down, with at least one port 257 being formed through the covering 258. The frame 265 may include a plurality of struts arranged as in a ring of a Z-stent, wherein the struts comprise a shape-memory material. The shape-memory material may be a polymer, or a nickel-titanium alloy, or any other material known in the art. The shape-memory frame 258 may be heat set to have a remembered state that is biased to an open configuration when the device is free from external compression. In some embodiments, the heat set temperature may be substantially body temperature, such that when the collection member 252 is deployed to a body vessel, it adopts the expanded configuration. Optionally, the collection member 252 may include outer lip 266 at its basal end.

Turning now to FIG. 5, a catheter system or assembly is illustrated. The catheter devices of FIGS. 1-4 may be used, in certain embodiments, as inner catheters of a catheter assembly. As illustrated in FIG. 5, catheter assembly 310 can include an outer catheter 322 and an inner catheter 324, each of which are generally tubular members having a lumen (328 and 371) extending therethrough. In some embodiments, outer catheter 322 and inner catheter 324 may be in a coaxial relationship, thereby forming an annular lumen 371 between an interior wall portion 326 of outer catheter 322 and an outer wall portion 343 of inner catheter 324, as shown in FIG. 5. To this end, the outer wall portion 350 of the outer catheter 322 generally defines the outer wall of the catheter system 310. The respective lumens of outer catheter 322 and inner catheter 324 can extend along the entire length of their respective tubular bodies. Further, the material of the collection member 352 can be configured to permit the collection member 352 to collapse and conform to the shape of the lumen of the outer catheter 322, in some embodiments filling the lumen 371 when retracted within the outer catheter 322.

In FIG. 5, inner catheter 324 is shown extending beyond the distal end 372 of outer catheter 322. The inner catheter 324 may be extended axially outward to ensure that the collection member 352 adopts the expanded configuration, which gives rise to the formation of the annular lumen 371 between the inner catheter 324 and the outer catheter 322. The inner catheter 324 can also be drawn proximally and axially into the outer catheter.

The inner and outer catheters 324/322 can be formed of a conventional polymer commonly used for such purposes in medical catheters, such as radiopaque polyurethane. Other conventional materials used for such purposes in the medical device art may be substituted. Non-limiting examples of such materials include polyether block amide, polyamide (nylon), silicone, polyurethane, and polytetrafluoroethylene (PTFE). Both catheters may comprise a multiple layered wall construction, with one of the layers being a reinforcement structural layer such as a coil and/or a braid to improve resistance to kinking, pushability, tractability, etc. For instance, to increase the column strength and pushability, the inner catheter may be constructed of FLEXOR® tubing construction, available from Cook, Inc (Bloomington, Ind.). Assembling of a multiple layered catheter wall is described in, e.g., U.S. Pat. No. 5,380,304 to Parker and U.S. Pat. No. 6,704,122 to Parker et al., each of which is incorporated herein by reference in its entirety.

The features described above can be supplemented with other known materials and techniques to improve various properties of the catheter assembly. For example, one or more radiopaque markers can be added along the length of the catheters, or a radiopaque material may be added to the matrix of all or a part of the catheters to improve visualization of the catheters in accordance with well-known techniques. Similarly, the catheters, and in particular the outer catheter, may include a hydrophilic coating along all or a part of the length of the catheter to facilitate entry into the vessel. As yet another alternative, the catheters can be coated or impregnated with various medicaments along all or a part of the length of the catheter body. Non-limiting examples of such medicaments include antiproliferatives, anticoagulants, thrombolytics, fibrinolytics, and antimicrobials.

FIG. 6 depicts an embodiment of the catheter system 410 in which the collection member 452 includes a sealing structure owing to outer lip 466. The outermost cross-sectional area of the outer lip 466 is sized to form a seal with the inner vessel wall of the vessel to be treated, such that the only fluid communication between the annular lumen 471 is via the at least one port 457. For instance, the outermost cross-sectional area of the outer lip 466 can be at least greater than the cross-sectional area of the outer catheter 422.

As shown in FIG. 6, the catheter system may further include a third catheter such as infusion catheter 464. This catheter 464 may extend through the lumen 471 of the outer catheter 422 and run to, or through, the port 457. The infusion catheter 464 may have tubular body and a lumen running therethrough, and may be in fluid communication with a reservoir located external the body of the patient such that an infusion fluid 474 may be flowed beyond the distal end of the catheter assembly 410 in order to disrupt a thrombus.

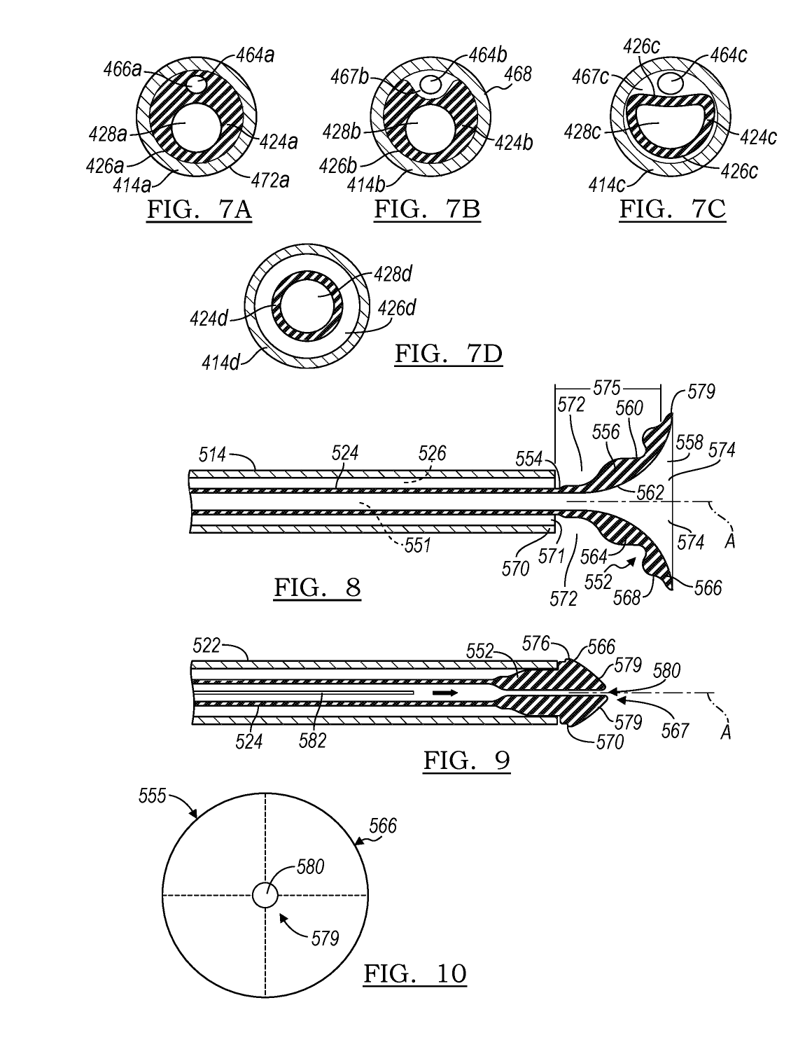

The inner catheter and the outer catheter of a system in accordance with the principles of the present invention may be constructed in a variety of ways. Turning now to FIGS. 7A-7D, the cross-sectional profiles of a number of catheter systems are displayed.

FIG. 7D portrays a cross-sectional alignment as most clearly illustrated in FIG. 5, wherein the wall 424d of the inner catheter is positioned such that the inner catheter is coaxial to the outer catheter (having wall 414d), such that an annular lumen 426d is formed between the two catheters. The inner lumen 428d of inner catheter 424d runs along the longitudinal axis of the device.

FIG. 7A is a cross-sectional profile of a device in accordance with another embodiment of the invention. In the device of FIG. 7A, the wall 424a of the inner catheter is formed to substantially fill the lumen 426a of the outer catheter, which is defined by wall 414a. Thus, no appreciable space exists between the inner catheter and the outer catheter, and lumen 428a represents the largest channel through the catheter system. Optionally, the wall 424a of the inner catheter has a second lumen 464a therethrough such that infusion catheter 466a, or in some embodiments a wire guide, may be run through the length of the catheter system.

FIG. 7B illustrates a cross-sectional profile of a device in accordance with another embodiment of the invention. In this case, the wall 424b of the inner catheter is thick and contacts a portion of the inner surface 426b of the wall 414b of the outer catheter, with a notch 467b formed in the profile of the wall 424b of the inner catheter. This allows for the infusion catheter 464b to run through a luminal space formed by notch 467b. Such a construction may be advantageous for uses in which it is desirable to run an infusion catheter through the system 410b after the other parts of the system have been deployed to the body vessel.

FIG. 7C shows the profile of another catheter system in accordance with the principles of the present invention. In this case, an inner catheter with a D-shaped lumen 428c and a wall 424c of substantially constant width is used as the inner catheter. This allows for contact between a portion of the outer wall of the inner catheter and the inner wall of the outer catheter, but also a luminal space 467c formed along the longer surface of the D-shaped catheter. This embodiment may allow for the placement of a larger infusion catheter 464c than does the embodiment of, for example, FIG. 7A or FIG. 7B.

In another embodiment, the present disclosure provides for a self-sealing catheter system, as is illustrated in FIGS. 8-10. In order to provide for such self-sealing, the collection member 552 of the device 510 is formed with certain countours in order to give rise to a number of advantageous properties of the device both in the expanded configuration 569 (FIG. 8) and in the collapsed configuration 568 (FIG. 9).

In the device 510, outer surface 560 of the collection member 552 may include a protruding region 564 which will facilitate collapse of the collection member 552 as will be described below. Inner surface 562 of the collection member 552 can be curved outward to facilitate funneling of emboli into the lumen 551 of inner catheter 524. Lumen 558 of the collection member 552 radially surrounds longitudinal axis A at an increasingly larger distance in the basal direction to provide for the funneling of thromboemboli as well. However, as will be appreciated by those skilled in the art, the collection member 552 may have at least a region of constant diameter (taking on a cup shape).

An outer lip 566 may be formed at the distal end of collection member 552. Outer lip 566 can extend radially outward to define the maximum radial extent of the collection member 552. Outer lip 566 may be further sized to sealably contact the vessel wall to inhibit migration of emboli anywhere other than into the lumen 551 of the collection catheter 524. To facilitate this sealing action, a curved outer surface 568 may be provided along outer lip 566. The combination of a flared wall of the collection member 552 and the outer lip 566 as shown in the figures can define a collection member 552 having a trumpet or bell shape.

Collection member 552 can be axially movable between an extended position and a retracted position. The extended position is shown in FIG. 8, where the collection member 552 has an open (or expanded) configuration 569. The retracted position is shown in FIG. 9, where the collection member 552 is moved to a collapsed configuration 563. Axial movement of collection member 552 can be affected by relative movement between inner catheter 524 and outer catheter 522, which can be controlled by an operator at the proximal portion of the catheter assembly 510.

In the extended position, collection member 552 is located distally beyond a distal end 570 of outer catheter 522. In particular, collection member 552 can be moved away from distal end 570 so that the collection member 552 can assume the open configuration 569. The distance 575 of extension of collection member 552 from distal end 570 of outer catheter 522 can be any distance sufficient to allow for thrombus collection and, optionally, effective infusion of fluid.

Optionally, in the expanded configuration 569 as shown in FIG. 8, the inner catheter 524 may be situated in lumen 526 of the outer catheter 522 such that an annular lumen 571 is formed. This space luminal space may be used for infusion of a fluid, either by flowing the fluid through the lumen 571 itself, or for the placement of an infusion catheter. The infused fluid would be infused distal the collection member into the body vessel via ports formed through the collection member 552 (not shown in this cross-sectional view.) The fluid infused may be, in some embodiments, saline, or another biocompatible buffer. In other embodiments, the infusion fluid may include a drug, such as a thrombolytic, which will assist in degrading and breaking down the thrombus into smaller, detached embolic particles.

In the retracted (collapsed) position 563 (FIG. 9), a substantial portion of collection member 552 can be situated within a portion of the lumen 526 of outer catheter 522 proximate distal end 570 to define the collapsed configuration 563. In the collapsed configuration 563, collection member 552 may advantageously take on the shape of an atraumatic tip 576. In some embodiments, the thickened portion 568 of the collection member 552 may function as a catch which prevents the collection member 552 to be drawn entirely into the lumen 526 of outer catheter 522. The tapered basal ridge 579 of the collection member 552 defines the extreme tip of the atraumatic tip formed from the collapsed collection member 552.

Advantageously, the collection member 552 may be constructed so that the collection member 552 collapses into, for example, quadrants 555, as shown in FIG. 10, which fold down so that they do not contact one another, instead forming channel 580 therethrough. Such a channel may be useful for passing a wire guide 582 (as shown in FIG. 9) through the length of the device, so that the wire guide 582 can be placed in the vasculature of the patient and the catheter system can be loaded over it. The wire guide lumen 580 may be coincident with longitudinal axis A in some embodiments.

The flexibility of the collection member 552 can facilitate closure thereof when moving from the expanded configuration and extended position to the collapsed configuration and the retracted position. For example, as collection member 552 is retracted into the lumen of outer catheter 522, the inner wall of the outer catheter 522 can slide along outer surface 560 of collection member 552. The wall 556 of the collection member 552 may include a thickened or protruding section 564 which can then be displaced radially inward toward axis A by contact with the inner wall of outer catheter 522.

Also, with reference to FIG. 9, the thickness of section 564 of the wall 556 of collection member 552 may be selected to fill the cross-sectional area of at least the lumen of outer catheter 522. The thickness of section 556 of the collection member 552 is generally greater than the wall thickness of the tubular portion of inner catheter 524. When present, protruding region 564 of collection member 552 can enhance closure of the collection member 552.

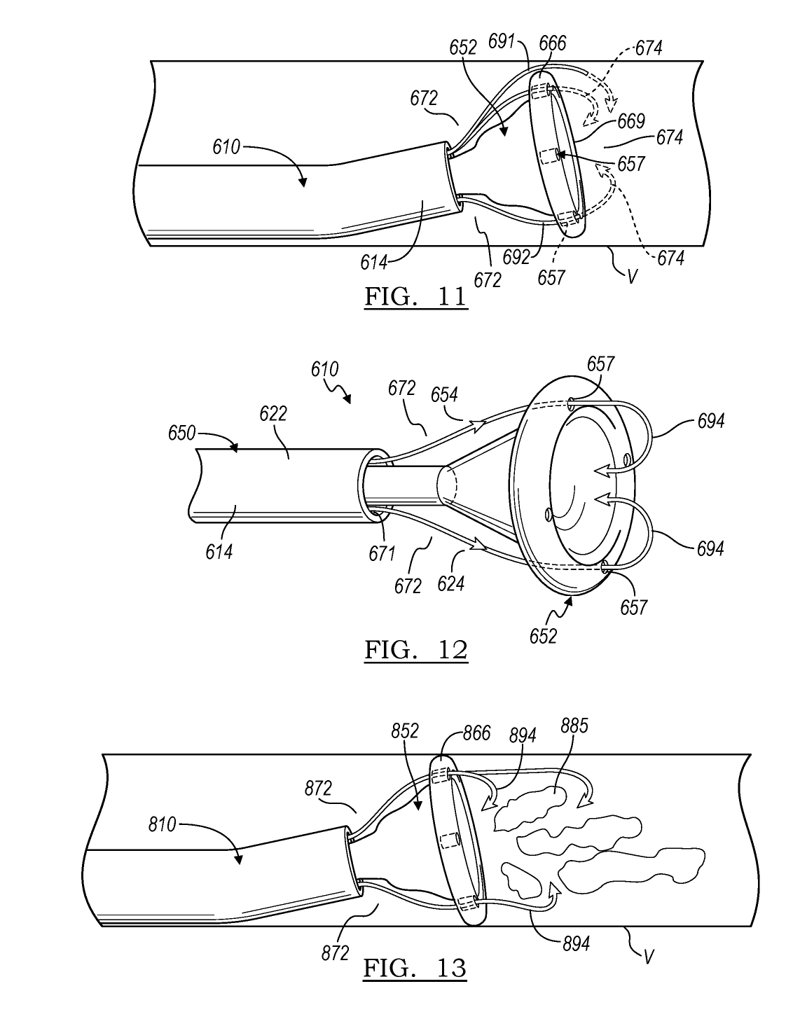

FIGS. 11 and 12 illustrate a view of a catheter assembly 610 in use in accordance with the principles of the present disclosure. As shown in FIG. 11, catheter system 610 is inserted in vessel V, with collection member 652 in its expanded state. A plurality of ports 657 are formed through the collection member 652. The presence of collection member 652 in the vessel V effectively divides the vessel into two different sections: a distal (thrombus-containing) section 674, and a proximal (non-thrombus-containing) section 672. The assembly 610 is shown with two infusion catheters 691/692. For illustration purposes, a number of different configurations are shown in FIG. 11. For example, first infusion catheter 691 is run through the annular lumen 671 between the inner catheter 624 and the outer catheter 622, and between the wall of vessel V and outer lip 666 of collection member 652. In contrast, second infusion catheter 692 runs through one of the infusion ports 657, infusing section 674 of vessel V with fluid to help disrupt a thrombus. In some embodiments, the distal end of infusion catheter 692 is contained within the port 657; in other embodiments, the infusion catheter 692 may represent the most distal portion of the system 610, extending through the port 657 into section 674 of the body vessel. In some embodiments, the infusion ports 657 may serve a plurality of functions, such as to introduce another tool to the vasculature therethrough. In one embodiment, the tool may be a wire guide or a scraping member, as described below. In another embodiment, the tool may comprise a basket, such as a filter-type basket, for placement between the end of the collection member 652 and the thrombus to be collected. The basket may be deployed through the port 657 in a collapsed state, and expanded when it is positioned between the collection member and the thrombus. The basket may be collapsed around thromboemboli released from the parent thrombus, and retracted from the vasculature with the remainder of the system.

FIG. 12 illustrates another principle of the device, namely that the annular lumen 671 can function as an infusion lumen. Flow lines 694 represent the flow of infusion fluid out lumen 671, through intravascular space 672, and through infusion ports 657, where the fluid emerges in vascular section 674 and disrupts the thrombus before being aspirated back through the lumen of collection member 652, proximally through the lumen of inner catheter 624, and out of the body. In such an embodiment, separate infusion catheters are not employed, as infusion fluid is driven from one side of the collection member 652 to the other due to a local buildup of fluid pressure in proximal section 672 of the vessel, expelling fluid through ports 657 to the distal section 674. FIG. 13 illustrates an embodiment similar to that of FIG. 12 in the process of being used to extract emboli. In this case, a lip 866 may be desirable for contacting and sealing against the wall of vessel V in order to ensure that infusion fluid is focused through the ports 854. Emboli 885, when disrupted, can then be aspirated into collection member 852 and removed from the vasculature safely.

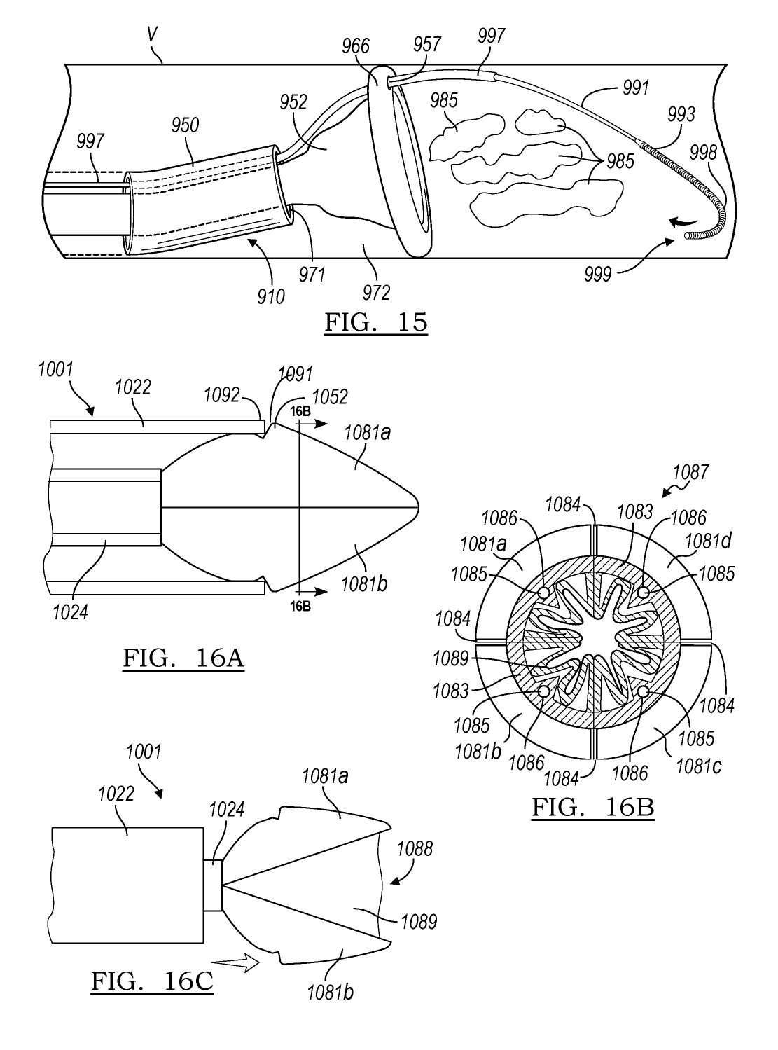

In another embodiment, it may be desirable to mechanically manipulate the thrombus to be removed prior to introduction of fluid or aspiration. One means of such manipulation may be a wire guide 991 as shown in FIG. 15. Here, the wire guide 991 is placed through a port 957 formed through the lip 966 of the collection member 952, optionally through a small catheter 997, in some embodiments about a 3 French catheter. In another embodiment, the port may be formed elsewhere in the collection member 952. The wire guide 991 in one embodiment may terminate in a hook 998 at end 999. The hook 998 can be brought into contact with the thrombus to be treated and can aid in the formation of thromboemboli 985. The wire guide 991 may taper distally, and may include a flex section 993 including a coil, disposed proximal to and including the hook 998. The taper may be proximal of the flex section 993, or distal the flex section 993, or both. The end 999 may be passed into or beyond the thrombus to be treated, and may be manipulated such as by rotating in order to break the thrombus up into smaller particles for removal.

In other embodiments, the end of the wire guide 991 may have another shape that is suitable for the manipulation of a thrombus, including but not limited to a corkscrew or spiral shape, a wedge shape, a brush, and so forth. In one embodiment, the wire guide 991 may simply be a standard wire guide without a modified or specialized end. In any embodiment, the wire guide may be used not only to manipulate the thrombus, but also to guide the system through the vasculature.

In another embodiment, as shown in FIGS. 16A-16C, the collapsed state 1087 of a device 1001 according to the principles of the present disclosure may be facilitated by the structure of the collection member 1052 including a membrane 1089. A device 1001 constructed in this way is seen in FIG. 16A, and FIG. 16B, which is a cross-sectional view taken along line 16B of FIG. 16A. The device 1001 of FIG. 16 is made of four supporting portions or quadrants 1081a/b/c/d, which may be separated by boundaries 1084. Each quadrant 1081 has a respective inner solid portion 1083, each having at least one cavity therein for the storage of a portion of membrane 1089. The collapsed condition of the membrane 1089 can be seen in FIG. 16B. Each inner solid portion 1083 may have a spring wire lumen 1086 formed therethrough and extending proximally through the collection member 1052. The spring wire lumen 1085 may be loaded with a shape memory wire 1085, such as one made of a nickel-titanium alloy, which is heat set such that the membrane 1089 (and the collection member 1052) is biased to have an open state (1088; see FIG. 16C). Such a bias will allow for easier expansion in the body vessel to be treated. As can be seen in FIG. 16C, when the inner catheter 1024 is advanced distally, the catch 1091 of the collection member 1052 is no longer engaged with the holding portion 1092 of the outer catheter 1022, and the collection member 1052 is expanded to the expanded state 1088, with membrane 1089 bridging the gaps between supporting portions 1081a/b/c/d.

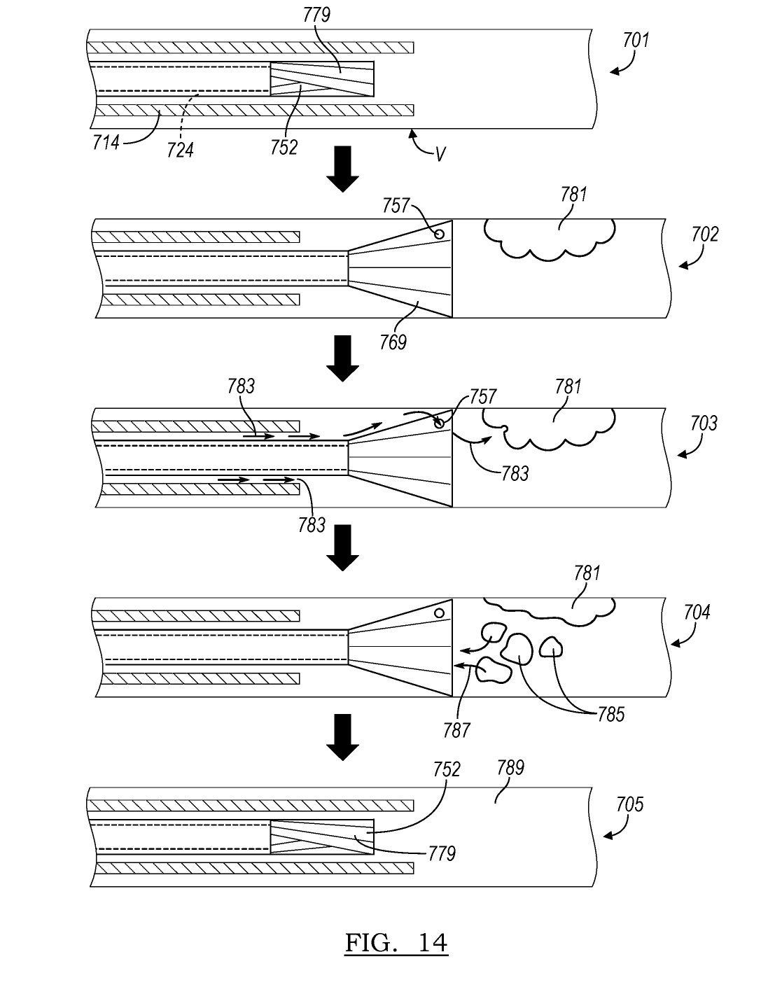

FIG. 14 provides a view of several steps in the extraction of a thrombus and clearance of emboli in accordance with one embodiment of the present invention. In a first step 701, catheter system 710 has been inserted into the vessel V. Insertion of the catheter assembly into the body vessel may be accomplished by any technique known in the art. Optionally, a distal portion of the catheter assembly can be inserted into the body vessel over a wire guide, such as via the well-known Seldinger percutaneous entry technique. The distal portion of the catheter body can be inserted into the vessel V with the collection member 752 in the collapsed and retracted position 779, and then translated to a site proximal a thrombus for treatment.

In a second step 702, the catheter assembly 710 has been moved just proximal the thrombus 781 to be extracted. The inner catheter 724 has been moved distally relative to the outer catheter 714, and because the collection member 752 is biased to its expanded configuration, such motion has resulted in its expansion to the expanded configuration 769. As illustrated, the collection member 752 makes contact with the wall of vessel V along its circumference, effectively forming a seal. However, this may be an optional feature in certain embodiments. In some embodiments, a tool such as a wire guide or a catch basket may be fed through the inner lumen of catheter 724 to physically disrupt the thrombus. Such a wire guide may have a disrupting structure, such as a zigzag configuration, to affect improved disruption. Such a disrupting tool would be used prior to aspiration.

In a third step 703, infusion fluid 783 flows through the annular lumen 771 in a distal direction, building up behind the outer surface of collection member 752 and causing fluid to flow through the infusion port 757. As illustrated, the system 710 does not comprise a separate infusion catheter, but one could be placed through the port to direct flow if desired. The infusion fluid flows through the port 757 and, either by the force of the flow or in conjunction with a thrombolytic agent, begins to disrupt the thrombus.

In fourth step 704, the thrombus 781 is reduced in size, breaking into thromboemboli 785. At this point, aspiration of the emboli through lumen of the collection member 752 commences, and the thrombus begins to be cleared from the vessel. Flow of infusion fluid 783 may also continue so that the thrombus 781 can be completely removed.

When the thrombus has been completely broken down and cleared from the vessel V, in fifth step 705, the inner catheter 724 is retracted and contact with the inner wall of outer catheter 722 causes collection member 752 to fold into the collapsed state 779. Site 789 is free of the thrombus.

Although the method of FIG. 14 was illustrated using a simple funnel shape for the collection member 752, a person of skill in the art will appreciate that the collection member 752 could readily have the shape of the device of FIG. 6 or of FIG. 8, among others.

Drawings in the figures illustrating various embodiments are not necessarily to scale. Some drawings may have certain details magnified for emphasis, and any different numbers or proportions of parts should not be read as limiting, unless so designated in the present disclosure. Those skilled in the art will appreciate that embodiments not expressly illustrated herein may be practiced within the scope of the present invention(s) of this disclosure, including those features described herein for different embodiments may be combined with each other and/or with currently-known or future-developed technologies while remaining within the scope of the claims presented here. It is therefore intended that the foregoing detailed description be regarded as illustrative rather than limiting. And, it should be understood that the following claims, including all equivalents, are intended to define the spirit and scope of this invention(s) of this disclosure.

1. A medical device for use in extraction of emboli from a body vessel, the medical device comprising:
a tubular member comprising a proximal end and extending to a distal end, the tubular member having a first lumen formed therethrough; and
a collection member having a collapsed configuration and an expanded configuration, the collection member being movable therebetween, the collection member having an apical end disposed to the distal end of the tubular member and extending to a basal end defining a body portion therebetween, the basal end being distal the apical end, the apical and basal ends having a second lumen formed therethrough and in fluid communication with the tubular member, the collection member comprising at least one port formed through the body portion.

2. The medical device of claim 1, wherein the body portion has a substantially conical shape in the expanded configuration.

3. The medical device of claim 1, wherein the collection member is biased to the expanded configuration.

4. The medical device of claim 1, wherein the collection member comprises a plurality of ports formed through the body portion and offset from the apical end.

5. The medical device of claim 1, wherein the body portion is radially collapsible to the collapsed position.

6. The medical device of claim 1, wherein the body portion comprises a lip toward the basal end.

7. The medical device of claim 6, wherein the lip comprises a plurality of protrusions such that in the collapsed configuration, the plurality of protrusions converge to form an atraumatic tip.

8. The medical device of claim 1, wherein the collection member defines a channel therethrough extending between the apical end and the basal end in the collapsed position, the channel being in fluid communication with the first lumen.

9. A medical device assembly for use in extraction of emboli from a body vessel, the assembly comprising:
an outer tubular member comprising a first proximal end and extending to a first distal end, the outer tubular member having a passageway extending therethrough and an opening at the first distal end;
an inner tubular member comprising a second proximal end and extending to a second distal end, the inner tubular member having a second lumen formed therethrough; and
a collection member having a collapsed configuration and an expanded configuration, the collection member being movable therebetween, the collection member having an apical end disposed at the second distal end of the inner tubular member and extending to a basal end defining a body portion therebetween, the basal end being distal the apical end, the apical and basal ends having a third lumen formed therethrough and in fluid communication with the inner tubular member, the collection member comprising at least one port formed through the body portion,
wherein the collection member is axially movable between a first position and a second position, the collection member being in the collapsed configuration and disposed within the outer tubular member in the first position, the basal end of the collection member being axially extended from the outer tubular member in the second position.

10. The medical device assembly of claim 9, wherein the body portion has a substantially conical shape in the expanded configuration.

11. The medical device assembly of claim 9, wherein the collection member is biased to the expanded configuration.

12. The medical device assembly of claim 9, wherein the inner tubular member is positioned within the passageway of the outer tubular member to define an annular lumen therebetween.

13. The medical device assembly of claim 9, wherein the inner tubular member is positioned within the passageway of the outer tubular member such that an outer surface of the inner tubular member is in contact with an inner surface of the outer tubular member for at least a portion of a length of the outer tubular member.

14. The medical device assembly of claim 9, wherein the collection member comprises a plurality of ports formed through the body portion and offset from the apical end.

15. The medical device assembly of claim 9, wherein the body portion is radially collapsible to the collapsed position.

16. The medical device assembly of claim 9, wherein the body portion comprises a lip toward the basal end.

17. The medical device assembly of claim 16, wherein the lip comprises a plurality of protrusions such that in the collapsed configuration, the plurality of protrusions converge to form an atraumatic tip, the lip being configured to rest distally against the first distal end of the outer tubular member while in the collapsed position.

18. The medical device assembly of claim 9, wherein the collection member defines a channel therethrough extending between the apical end and the basal end in the collapsed position, the channel being in fluid communication with the lumen of the tubular member.

19. The medical device assembly of claim 18, further comprising a wire guide.

20. The medical device assembly of claim 19, wherein the wire guide is disposed in the channel.

21. The medical device assembly of claim 19, wherein the wire guide is disposed through a port.

22. The medical device assembly of claim 21, wherein the wire guide comprises a distal end having a hook.

23. The medical device assembly of claim 9, further comprising an infusion catheter, the infusion catheter having a tubular body and extending through the passageway of the outer tubular member and through a port of the collection member.
